(19) United States
(12) Patent Application Publication
(10) Pub. No.: US 2010/0297528 A1
(43) Pub. Date: Nov. 25, 2010

(54) ALKYLATED BISPHENOL-BASED COMPOUND AND PREPARATION, SULFONATED POLYARYLENE SULFONE POLYMER PREPARED FROM THE COMPOUND, AND FUEL CELL USING THE POLYMER

(75) Inventors: Yeong-suk Choi, Suwon-si (KR); Sang-ouk Kim, Daejeon (KR); Won-jun Lee, Daejeon (KR); Sun-hwa Lee, Daejeon (KR)

Correspondence Address: STEIN MCEWEN, LLP, 1400 EYE STREET, NW, SUITE 300, WASHINGTON, DC, 20005, US

(73) Assignees: Samsung Electronics Co., Ltd., Suwon-si (KR); Korea Advanced Institute of Science and Technology, Daejeon (KR)

(21) Appl. No.: 12/771,280

(22) Filed: Apr. 30, 2010

(30) Foreign Application Priority Data
May 19, 2009 (KR) 10-2009-0043591

(51) Int. Cl. H01M 8/10; C07C 39/16; C08J 5/20

(52) U.S. Cl. 429/492; 568/723; 521/25

(57) ABSTRACT

An alkylated bisphenol-based compound, a method of preparing the same, sulfonated polyarylene sulfone polymer prepared from the alkylated bisphenol-based compound, a method of preparing the polymer, and a fuel cell using the sulfonated polyarylene sulfone polymer.

### CROSS-REFERENCE TO RELATED APPLICATION

This application claims the benefit of Korean Application No. 10-2009-0043591, filed May 19, 2009 in the Korean Intellectual Property Office, the disclosure of which is incorporated herein by reference.

### BACKGROUND

1. Field

One or more embodiments of the present invention relate to an alkylated bisphenol-based compound, a method of preparing the same, a sulfonated polyarylene sulfone prepared from the alkylated bisphenol-based compound, and a fuel cell using the sulfonated polyarylene sulfone.

2. Description of the Related Art

Fuel cells may be classified into polymer electrolyte membrane fuel cells (PEMFCs), phosphoric acid fuel cells, molten carbonate electrolyte fuel cells, and solid oxide fuel cells, according to the type of electrolyte used. A PEMFC includes a cathode, an anode, and a polymer electrolyte membrane interposed between the cathode and the anode. The anode includes a catalyst layer for catalyzing the oxidation of a fuel. The cathode includes a catalyst layer for catalyzing the reduction of an oxidant.

In a PEMFC, the polymer electrolyte membrane serves not only as an ion conductor for the transfer of protons from the anode to the cathode, but also as a separator to block physical contact of the anode and the cathode. Thus, the properties required of a polymer electrolyte membrane include excellent ion conductivity, electrochemical stability, high mechanical strength, thermal stability at operating temperatures, and the ability to be easily formed into a thin film. Polymer electrolyte membranes may be formed of sulfonated polysulfone polymers. However, the mechanical properties of sulfonated polysulfone polymers are currently inadequate.

### SUMMARY

One or more embodiments of the present invention include an alkylated bisphenol-based compound, a method of preparing the same, a sulfonated polyarylene sulfone prepared from the alkylated bisphenol-based compound, and a fuel cell using the sulfonated polyarylene sulfone.

According to one or more embodiments of the present invention, an alkylated bisphenol-based compound is represented by Formula 1.

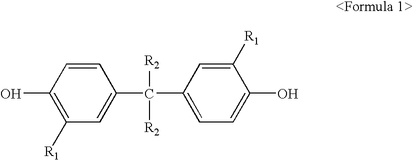

where R₁ is a C1 to C30 alkyl group and

R₂ is a hydrogen atom or a C1 to C5 alkyl group.

According to one or more embodiments of the present invention, a sulfonated polyarylene sulfone includes a first repeating unit represented by Formula 3 and a second repeating unit represented by Formula 4:

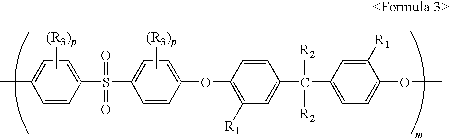

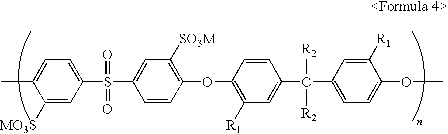

where R₁ is a C1 to C30 alkyl group,

R₂ is a hydrogen atom or a C1 to C5 alkyl group,

each R₃ is the same as or different from other R₃'s and is independently a C1-C10 alkyl group, a C2-C10 alkenyl group, a phenyl group or a nitro group,

p is an integer from about 0 to about 4,

M is sodium (Na), potassium (K), or hydrogen (H), and

m is from about 0.01 to about 0.99, and n is from about 0.01 to about 0.99.

According to one or more embodiments of the present invention, a method of preparing an alkylated bisphenol-based compound represented by Formula 1 includes addition of C2-C30 alkenes to a compound represented by Formula 6 below to obtain the alkylated bisphenol-based compound.

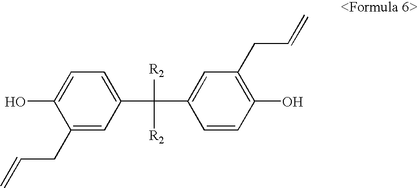

where R₂ is a hydrogen atom or a C1 to C5 alkyl group,

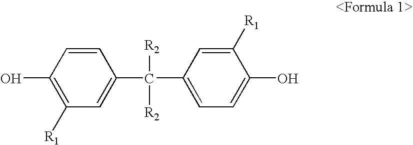

where R₁ is a C1 to C30 alkyl group, and

R₂ is a hydrogen atom or a C1 to C5 alkyl group.

According to one or more embodiments of the present invention, a method of preparing a sulfonated polyarylene sulfone including a first repeating unit represented by Formula 3 below and a second repeating unit represented by Formula 4 below includes polymerization of an alkylated bisphenol-based compound represented by Formula 1 below, a compound represented by Formula 7 below, and a compound of Formula 8 below.

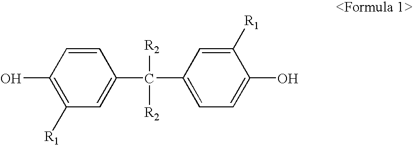

where R₁ is a C1 to C30 alkyl group and

R₂ is a hydrogen atom or a C1 to C5 alkyl group.

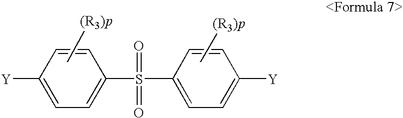

where each R₃ is the same as or different from other R₃'s and is a C1-C10 alkyl group, a C2-C10 alkenyl group, a phenyl group or a nitro group,

p is an integer from about 0 to about 4, and

Y is chlorine (Cl), fluorine (F), bromine (Br), or iodine (I).

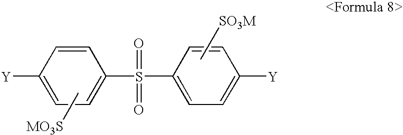

where M is sodium (Na), potassium (K), or hydrogen (H) and

Y is chlorine (Cl), fluorine (F), bromine (Br), or iodine (I).

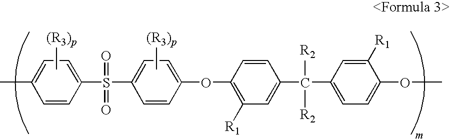

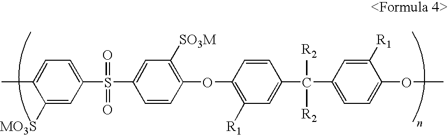

where R₁ is a C1 to C30 alkyl group,

R₂ is a hydrogen atom or a C1 to C5 alkyl group,

each R₃ is the same as or different from other R₃'s and is a C1-C10 alkyl group, a C2-C10 alkenyl group, a phenyl group or a nitro group,

p is an integer from about 0 to about 4,

M is sodium (Na), potassium (K), or hydrogen (H), and

m is from about 0.01 to about 0.99, and n is from about 0.01 to about 0.99, provided that m+n=1.

According to one or more embodiments of the present invention, a fuel cell includes a cathode, an anode, and an electrolyte membrane disposed between the cathode and the anode, wherein the electrolyte membrane includes the sulfonated polyarylene sulfone described above.

Additional aspects and/or advantages of the invention will be set forth in part in the description which follows and, in part, will be obvious from the description, or may be learned by practice of the invention.

### BRIEF DESCRIPTION OF THE DRAWINGS

These and/or other aspects will become apparent and more readily appreciated from the following description of the embodiments, taken in conjunction with the accompanying drawings of which:

FIG. 1 illustrates nuclear magnetic resonance (NMR) spectra of diundecyl bisphenol A, dipentadecyl bisphenol A, and ditricosyl bisphenol A obtained according to Synthesis Example 1;

FIGS. 2 and 3 illustrate NMR spectra of sulfonated polyarylene sulfones obtained according to Examples 10 and 11;

FIGS. 4 and 5 are graphs illustrating the results of analyzing sulfonated polyarylene sulfones prepared according to Examples 16 and 19, respectively, using differential scanning calorimetry (DSC);

FIG. 6 is a graph illustrating the results of measuring the modulus of sulfonated polyarylene sulfones prepared according to Examples 11, 12 and 16;

FIG. 7 illustrates diffusion ordered spectroscopic (DOSY) spectra of sulfonated polyarylene sulfone prepared according to Example 16 before and after polymerization;

FIG. 8 is a perspective exploded view of a fuel cell according to an embodiment of the present invention; and

FIG. 9 is a cross-sectional view of a membrane-electrode assembly (MEA) of FIG. 8.

### DETAILED DESCRIPTION

Reference will now be made in detail to the present embodiments of the present invention, examples of which are illustrated in the accompanying drawings. The embodiments are described below in order to explain the present invention by referring to the figures.

One or more embodiments of the present invention include an alkylated bisphenol-based compound represented by Formula 1.

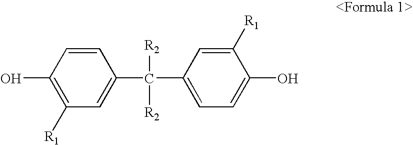

where R₁ is a C1 to C30 alkyl group and

R₂ is a hydrogen atom or a C1 to C5 alkyl group.

The alkylated bisphenol-based compound of Formula 1 may be hydrophobic depending on the type of group represented by R₁. According to the present embodiment, R₁ may be a C8-C23 alkyl group.

The alkylated bisphenol-based compound may be formed by a reaction of a diallyl bisphenol A based compound represented by Formula 6 below with an alkene.

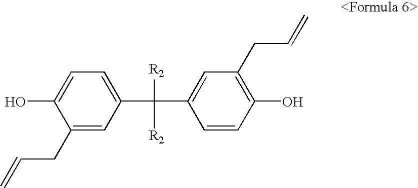

where R₂ is a hydrogen atom or a C1 to C5 alkyl group.

The above reaction is an addition reaction and may be implemented using a radical initiator like those used in radical polymerization. The radical initiator is used to connect the double bond between an allyl group of diallyl bisphenol A and an alkenyl group of an alkene, so that the alkylated bisphenol-based compound of Formula 1 may be obtained. When various alkenes are introduced to diallyl bisphenol A, various dialkyl bisphenols A that are proportional to the chain length of alkene may be obtained.

The alkylated bisphenol-based compound may produce a polymer having ionic conductivity such as sulfonated polyarylene sulfone or poly(etheretherketone) by condensation polymerization. Here, due to an alkyl group of the alkylated bisphenol-based compound of Formula 1, when the alkylated bisphenol-based compound of Formula 1 is used to form an ion conducting polymer such as sulfonated polyarylene sulfone, the glass transition temperature of the ion conducting polymer may be decreased and hydrophobicity of the ion conducting polymer may be increased so that resistance to water of the ion conducting polymer may be increased.

According to the present embodiment, R₁ in Formula 1 may be undecyl (—(CH₂)₁₀CH₃), pentadecyl (—(CH₂)₁₄CH₃), heneicosyl (—(CH₂)₂₀CH₃) or tricosyl (—(CH₂)₂₂CH₃). That is, according to the present embodiment, the alkylated bisphenol-based compound of Formula 1 may be a compound represented by Formula 2.

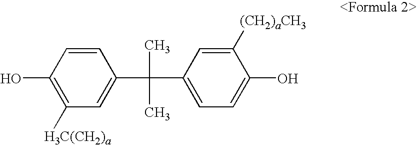

where a is an integer from about 4 to about 22.

That is, according to the present embodiment, a in Formula 2 may be an integer of 10, 14, 20 or 22.

One or more embodiments of the present invention include sulfonated polyarylene sulfone. The sulfonated polyarylene sulfone is formed by using the alkylated bisphenol-based compound of Formula 1 and is a copolymer including a first repeating unit represented by Formula 3 below and a second repeating unit represented by Formula 4 below.

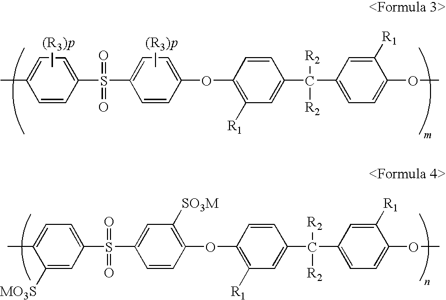

where R₁ is a C2 to C30 alkyl group,

R₂ is a hydrogen atom or a C1 to C5 alkyl group,

each R₃ is the same as or different from other R₃'s and is a C1-C10 alkyl group, a C2-C10 alkenyl group, a phenyl group or a nitro group,

p is an integer from about 0 to about 4,

M is sodium (Na), potassium (K), or hydrogen (H), and

m is from about 0.01 to about 0.99, and n is from about 0.01 to about 0.99.

In Formulae 3 and 4, m is a mixing ratio of the first repeating unit and n is a mixing ratio of the second repeating unit. According to the present embodiment, m may be from about 0.4 to about 0.9, and n may be from about 0.1 to about 0.6. The sulfonated polyarylene sulfone may have a degree of polymerization (DP) of about 5 to about 3,500. The group represented by (R₃)ₚ is hydrogen when p is 0.

According to an embodiment of the present invention, the sulfonated polyarylene sulfone may be a compound represented by Formula 5.

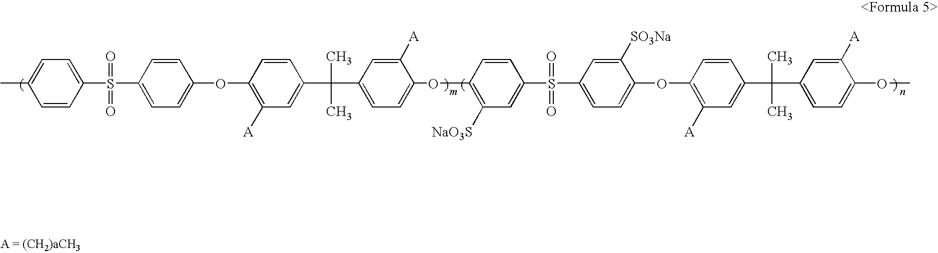

where m is from about 0.01 to about 0.99, n is from about 0.01 to about 0.99, the degree of polymerization is about 5 to about 3500, and a is an integer from about 4 to about 22.

According to the present embodiment of the present invention, m may be from about 0.4 to about 0.9, and n may be from about 0.1 to about 0.6. The sulfonated polyarylene sulfone has a degree of sulfonation of about 10 to about 50%.

According to an embodiment of the present invention, the sulfonated polyarylene sulfone may further include at least one selected from the group consisting of a third repeating unit represented by Formula 3a and a fourth repeating unit represented by Formula 4a.

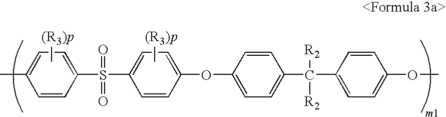

where R₂ is a hydrogen atom or a C1 to C5 alkyl group,

each R₃ is the same as or different from other R₃'s and is a C1-C10 alkyl group, a C2-C10 alkenyl group, a phenyl group or a nitro group,

p is an integer from about 0 to about 4, and

m1 is from about 0.01 to about 0.99.

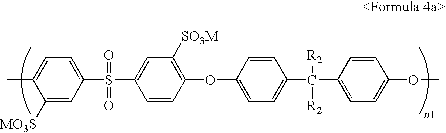

where R₂ is a hydrogen atom or a C1 to C5 alkyl group, and

n1 is from about 0.01 to about 0.99.

According to the present embodiment, m1 may be from about 0.4 to about 0.9 and n1 may be from about 0.1 to about 0.6.

If the sulfonated polyarylene sulfone only contains the first repeating unit of Formula 3 and the second repeating unit of Formula 4, the degree of sulfonation may be represented as [n/(m+n)]×100. If the sulfonated polyarylene sulfone contains first, second, third, and fourth repeating units respectively represented by Formulas 3, 4, 3a, and 4a, the degree of sulfonation may be represented as [(n+n1)/(m+m1+n+n1)]×100. The range of the degree of sulfonation may be about 10% to about 50%. When the degree of sulfonation of the sulfonated polyarylene sulfone is in the above range, a membrane-electrode assembly (MEA) including the sulfonated polyarylene sulfone may have high performance.

According to an embodiment of the present invention, if the sulfonated polyarylene sulfone includes the third repeating unit of Formula 3a and the fourth repeating unit of Formula 4a, a bisphenol-based compound represented by Formula 9 may be used as a polymerizable monomer.

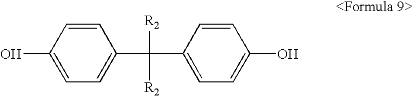

where R₂ is a hydrogen atom or a C1 to C5 alkyl group.

Sulfonated polyarylene sulfone according to an embodiment of the present invention may further use diallyl bisphenol A represented by Formula 6 being a diol compound as a polymerizable monomer and may further include a repeating unit formed therefrom.

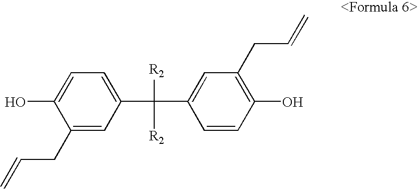

where R₂ is a hydrogen atom or a C1 to C5 alkyl group.

Another embodiment of the present invention provides sulfonated polyarylene sulfone or a cross-linked product thereof and a clay-sulfonated polyarylene sulfone nanocomposite including non-modified clay which is dispersed in the sulfonated polyarylene sulfone or a cross-linked product of sulfonated polyarylene. The cross-linked product of the sulfonated polyarylene sulfone may be a product obtained by a cross-linking reaction of the sulfonated polyarylene sulfone or a product obtained by a cross-linking reaction of sulfonated polyarylene sulfone with a multi-functional compound.

The multi-functional compound may be a monomer having a bi-functional group (for example, having two or more double bonds) with a low softening point. When the multi-functional compound is added in the cross-linking reaction and is UV hardened, the cross-linked product of sulfonated polyarylene sulfone having various structures may be obtained. Examples of the multi-functional compound may include bisphenol-A ethoxylate diacrylate, triethyleneglycol divinyl ether, and 1,6-hexanediol diacrylate.

The amount of the multifunctional compound may be in the range of about 0.1 to about 100 parts by weight based on 100 parts by weight of the sulfonated polyarylene sulfone. When the amount of the multi-functional group is in the above range, ionic conductivity of the finally obtained sulfonated polyarylene sulfone may be improved. The crosslinking reaction of sulfonated polyarylene sulfone with a crosslinking agent may be accomplished by irradiating with light in the presence of a polymerization initiator.

The polymerization initiator may include at least one selected from the group consisting of benzoyl peroxide and benzophenone. The amount of the polymerization initiator may be in the range of about 0.01 to about 10 parts by weight based on 100 parts by weight of the sulfonated polyarylene sulfone, for example, about 0.5 to about 5 parts by weight.

Another embodiment of the present invention provides a clay-sulfonated polyarylene sulfone nanocomposite formed of the sulfonated polyarylene sulfone or a cross-linked product thereof and clay. The nanocomposite may further include at least one selected from the group consisting of SiO₂ and TiO₂ in addition to the clay.

In the nanocomposite, the clay has a layered structure and is uniformly dispersed in the sulfonated polyarylene sulfone or a cross-linked product thereof, and the sulfonated polyarylene sulfone or a cross-linked product thereof is intercalated between layers of the clay. In some cases, the interlayer distance between layers of the clay is increased so that the layers may be exfoliated.

The nanocomposite according to the present embodiment of the present invention in which the layers of the clay are dispersed in a polymer while the sulfonated polyarylene sulfone having high ionic conductivity is intercalated or exfoliated between the layers has excellent mechanical properties, solvent resistance, brittleness, and ionic conductivity. The mechanical properties of the clay-sulfonated polysulfone nanocomposite may be maintained even when the polysulfone is highly sulfonated.

The clay refers to a layered silicate, wherein gaps between the layers thereof are expanded by water or an intercalating agent. The clay is formed using a simpler process than that of modified clay reformed with an organic phosphonium, an alkyl ammonium, or the like, thereby increasing manufacturing efficiency and reducing costs. In addition, the clay has more affinity to water than to methanol. When the clay is dispersed on a nanoscale scale in the membrane in an exfoliated form or in an intercalated form, even a small amount of the clay may suppress methanol crossover. In addition, the absorptivity of the clay may also minimize the reduction in membrane conductivity caused by the addition of an inorganic material.

The amount of the clay may be about 0.01 to about 50 parts by weight based on 100 parts by weight of the nanocomposite. When the amount of the clay is in the above range, barrier properties of the nanocomposite are improved without increasing viscosity and brittleness of the nanocomposite.

According to the present embodiment, the non-modified clay may be a smectite-based clay. Examples of the smectite-based clay may include montmorillonite, bentonite, saponite, beidellite, nontronite, hectorite, and stevensite.

The clay nanocomposite according to the present embodiment has the clay having a layered structure not only uniformly dispersed within the sulfonated polyarylene sulfone, but also present in an exfoliated form. In some cases, the interlayer distance between layers of the clay is increased so that the sulfonated polyarylene sulfone may be intercalated between the layers.

The nanocomposite according to the present embodiment in which the layers of the clay having a layered structure are dispersed in the sulfonated polyarylene sulfone or a cross-linked product thereof while the sulfonated polyarylene sulfone having high ionic conductivity is intercalated or exfoliated between the layers has excellent mechanical strength, heat resistance, and ionic conductivity. Also, when the nanocomposite is soaked with water, the intrusion of polar organic fuels such as methanol and ethanol, into the nanocomposite is suppressed. Since the nanocomposite may suppress the crossover of the polar organic fuel, the nanocomposite may be used to form an electrolyte membrane of a fuel cell in which a polar organic fuel cell is directly provided to an anode.

Hereinafter, methods of preparing dialkyl bisphenol A, sulfonated polyarylene sulfone, a cross-linked product of the sulfonated polyarylene sulfone, and a clay-sulfonated polyarylene sulfone nanocomposite using the same will be described. The alkylated bisphenol-based compound of Formula 1 above may be obtained by mixing a C2-C30 alkene with the diallyl bisphenol-based compound of Formula 6 below and polymerizing the mixture by applying light or heat to the mixture.

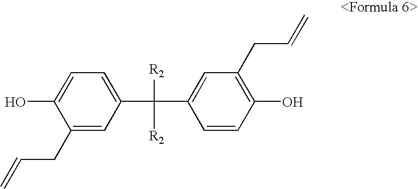

where R₂ is a hydrogen atom or a C1 to C5 alkyl group.

Examples of the C2-C30 alkene may include 1-octene, 1-dodecene, 1-octadecene, and 1-eicosene. The amount of the alkene may be about 0.5 to about 3 moles based on 1 mole of the diallyl bisphenol-based compound.

The polymerization is performed by radical polymerization in the presence of a radical initiator by applying heat or light. The light may be UV light. When heat is applied, the reaction temperature is maintained in a range of about 40 to about 90° C.

The radical initiator may be azobisisobutyronitrile (AIBN), benzyl peroxide, or the like and the amount of the radical initiator may be in the range of about 0.001 to about 2 moles based on 1 mole of diallyl bisphenol A. When the amount of the radical initiator is in the above range, reactivity of the radical reaction may be high. While the diallyl bisphenol A of Formula 6 and the C2-C30 alkene are polymerized, a product a by-product may be formed by ionic-bonding of the diallyl bisphenol A and the radical initiator, in addition to dialkyl bisphenol A.

Next, a synthesis of sulfonated polyarylene sulfone from the alkylated bisphenol-based compound will be described. First, the sulfonated polyarylene sulfone according to an embodiment of the present invention may be obtained by mixing a polymerizable monomer of Formula 7 below, a polymerizable monomer of Formula 8 below, and the diol form of alkylated bisphenol-based compound of Formula 1 and polymerizing the mixture in a solvent and a base. The polymerization is performed under a chemically inactive gas atmosphere such as nitrogen gas.

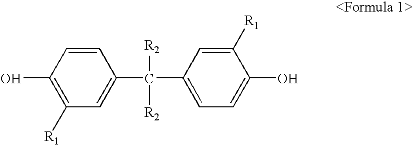

where R₁ is a C1 to C30 alkyl group and

R₂ is a hydrogen atom or a C1 to C5 alkyl group.

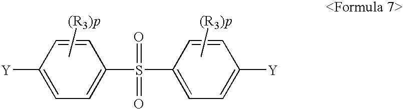

where each R₃ is the same r or different from other R₃'s and is a C1-C10 alkyl group, a C2-C10 alkenyl group, a phenyl group or a nitro group,

p is an integer from about 0 to about 4, and

Y is chlorine (Cl), fluorine (F), bromine (Br), or iodine (I).

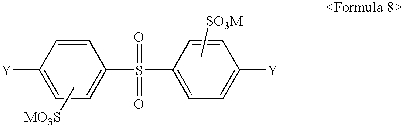

where M is sodium (Na), potassium (K), or hydrogen (H),

Y is chlorine (Cl), fluorine (F), bromine (Br), or iodine (I), and

R2 is a hydrogen atom or a C1 to C5 alkyl group.

The polymerization reaction is a condensation polymerization, and potassium carbonate (K₂CO₃) may be used as a base. The amount of the base may be in the range of about 0.1 to about 3 moles based on 1 mole of the alkylated bisphenol-based compound of Formula 1.

A solvent of the polymerization reaction may be N-methylpyrrolidone, dimethyl formamide, dimethyl sulfoxide, or the like. The amount of the solvent may be about 150 to about 700 parts by weight based on 100 parts by weight of the alkylated bisphenol-based compound of Formula 1.

A bisphenol-based compound represented by Formula 9 may be further added during the polymerization.

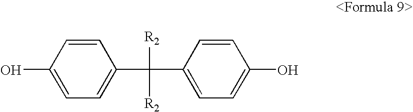

where R₂ is a hydrogen atom or a C1 to C5 alkyl group.

According to an embodiment, R₂ in Formula 9 may be a methyl group. As such, when the diol compound of Formula 9 participates in the polymerization, the sulfonated polyarylene sulfone finally obtained further includes at least one selected from the group consisting of the third repeating unit represented by Formula 3a above and the fourth repeating unit represented by Formula 4a above.

In addition, the diallyl bisphenol-based compound of Formula 6 below may be further added during the polymerization.

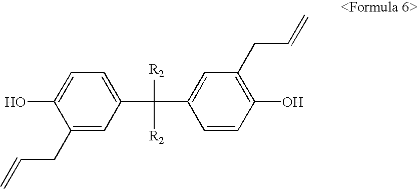

where R₂ is a hydrogen atom or a C1 to C5 alkyl group.

As described above, when the diallyl bisphenol-based compound of Formula 6 participates in the polymerization, the sulfonated polyarylene sulfone finally obtained may further include a repeating unit formed therefrom.

Examples of the polymerizable monomer represented by Formula 7 may include 4,4′-dichlorodiphenyl sulfone (DCDPS), 4,4′-difluorodiphenyl sulfone, and the like. Examples of the polymerizable monomer represented by Formula 8 may include sulfated-4,4′-dichlorodiphenyl sulfone (s-DCDPS), and the like.

The base may be potassium carbonate (K₂CO₃). The amount of the base may be in the range of about 0.1 to about 3 moles based on 1 mole of the alkylated bisphenol-based compound of Formula 1.

The polymerization may be performed at a temperature at which water generated during a nucleophilic reaction is refluxed with toluene and is removed, and may be performed at a temperature of about 100 to about 190° C. Subsequently, the polymerization product is cooled and then subjected to a work-up process, such as precipitation using isopropyl alcohol (IPA) or distilled water, to obtain sulfonated polyarylene sulfone.

The amount of the polymerizable monomer of Formula 8 may be in the range of about 20 to about 60 moles based on 100 moles of the polymerizable monomer of Formula 7. When the amount of the polymerizable monomer of Formula 8 is in the above range, the ionic conductivity of an electrolyte membrane may be increased and the electrolyte membrane may be easily formed because of avoidance of swelling by water.

The amount of the alkylated bisphenol-based compound of Formula 1 may be in the range of about 1 to about 100 moles based on 100 moles in total of the polymerizable monomer of Formula 7 and the polymerizable monomer of Formula 8. When the amount of the alkylated bisphenol-based compound of Formula 1 is not included in the above range, reactivity of the polymerization may be insufficient.

When the bisphenol-based compound of Formula 9 is further added, in addition to the alkylated bisphenol-based compound of Formula 1, the amount of the bisphenol-based compound of Formula 9 may be in the range of about 0.1 to about 0.95 moles based on 1 mole of the alkylated bisphenol-based compound of Formula 1.

The cross-linked product of the sulfonated polyarylene sulfone may be obtained by dissolving the sulfonated polyarylene sulfone in a solvent, adding the photopolymerization initiator thereto, and irradiating the mixture with light to photopolymerize. In the alternative, the cross-linked product may be obtained by dissolving the sulfonated polyarylene sulfone and the photopolymerization initiator in the solvent, adding the multifunctional compound such as hexanediol diacrylate thereto, irradiating the mixture with light to photopolymerize. Here, the order of the addition does not affect the properties of the cross-linked product.

A common type and amount of the photopolymerization initiator may be used. The photopolymerization initiator may be benzoyl peroxide, benzophenone, or the like. The amount of the photopolymerization initiator may be in the range of about 0.1 to about 5 parts by weight based on 100 parts by weight of the sulfonated polyarylene sulfone.

Hereinafter, a method of preparing a clay-sulfonated polyarylene sulfone nanocomposite using sulfonated polyarylene sulfone or a cross-linked product thereof, according to an embodiment of the present invention, will be described. The sulfonated polyarylene sulfone or a cross-linked product thereof according to an embodiment of the present invention is dissolved in a solvent by using a simple solution dispersion method and then a clay dispersion solution obtained by dispersing a non-modified clay in a dispersion medium is added thereto. The resultant mixture is vigorously stirred at room temperature, approximately 20° C., for about 6 to about 48 hours, for example, for about 24 hours. The solvent may be dimethyl acetamide (DMAc), N-methylpyrrolidone (NMP), dimethyl formamide (DMFA), dimethyl sulfoxide (DMSO), or the like. The amount of the solvent may be about 100 to about 600 parts by weight based on 100 parts by weight of the sulfonated polyarylene sulfone or the cross-linked product thereof.

Alternatively, the polymerizable monomer that is used to form the sulfonated polyarylene sulfone and potassium carbonate are used, and water and toluene therein are removed. Then, a nucleophilic reaction is performed at a temperature in the range of about 100 to about 190° C. so as to synthesize a polymer and the temperature of the reactor is reduced to about 70° C. Next, clay that is previously dispersed in a solvent for polymerization (for example, clay/NMP=2 g/50 g) is injected into the reactor, and the resultant mixture is stirred for about 12 hours or more, precipitated, and collected. Thus, the nanocomposite may be manufactured.

According to the present embodiment, the polymerization temperature of the sulfonated polyarylene sulfone, in which the polymerization is completed, is reduced to about 70° C., the clay that is previously dispersed in a solvent such as N-methylpyrrolidone is added and stirred, a non-miscible solvent is used to form a precipitate, the formed precipitate is washed using water, and thus clay-sulfonated polyarylene sulfone nanocomposite may be manufactured.

The sulfonated polyarylene sulfone, the cross-linked product thereof or the polymer in the clay-sulfonated polyarylene sulfone nanocomposite may have a weight average molecular weight of about 20,000 to about 3,500,000, and a number average molecular weight of about 10,000 to about 1,700,000. When the weight average molecular weight and the number average molecular weight thereof are respectively in the above ranges, film formation properties and processability are excellent.

A method of forming a nanocomposite electrolyte membrane for a fuel cell using the sulfonated polyarylene sulfone or a nanocomposite thereof, according to an embodiment of the present invention, will be described below in detail. A composition for forming the nanocomposite electrolyte membrane obtained by mixing the sulfonated polyarylene sulfone or the nanocomposite obtained as above and a solvent is used to form the nanocomposite electrolyte membrane by using casting or coating. The solvent may be dimethylacetamide (DMAc). The amount of the solvent may be in the range of about 150 to about 700 parts by weight based on 100 parts by weight of the nanocomposite. When the amount of the solvent is in the above range, casting or coating may be easily performed and mechanical properties of the nanocomposite electrolyte membrane are excellent. The sulfonated polyarylene sulfone having a hydrophobic group is used to form the nanocomposite electrolyte membrane manufactured according to the present embodiment so that swelling and chemical stability of the membrane are improved.

The thickness of the nanocomposite electrolyte membrane manufactured according to the current embodiment of the present invention is not limited. However, when the thickness of the nanocomposite electrolyte membrane is in a proper range, the strength of the nanocomposite electrolyte membrane may increase without an increase in internal resistance of a fuel cell including the nanocomposite electrolyte membrane. In this regard, the nanocomposite electrolyte membrane may have a thickness of about 10 to about 200 μm.

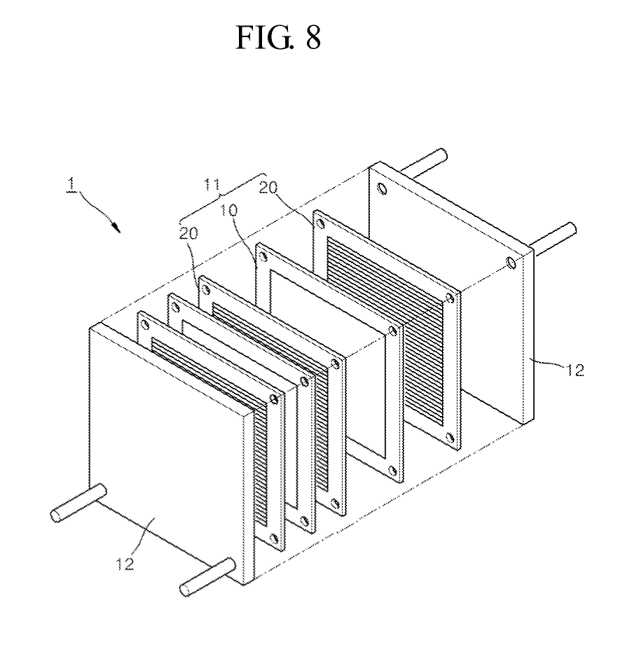

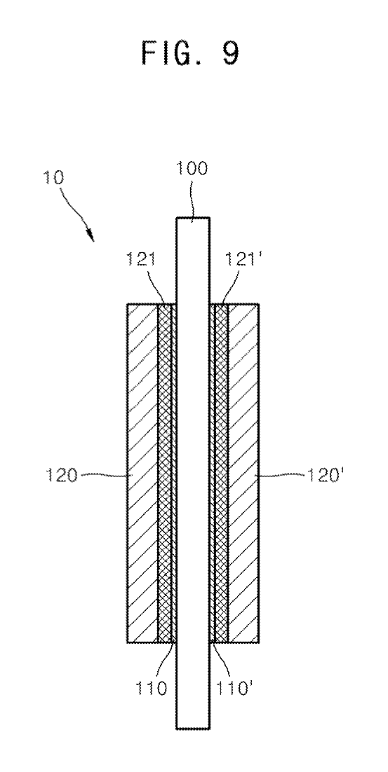

FIG. 8 is a perspective exploded view of a fuel cell 1 according to an embodiment of the present invention, and FIG. 9 is a cross-sectional view of a membrane-electrode assembly (MEA) of the fuel cell 1 of FIG. 8.

Referring to FIG. 8, the fuel cell 1 according to the present embodiment includes two unit cells 11 interposed between a pair of holders 12. Each unit cell 11 includes an MEA 10, and a pair of bipolar plates 20 respectively disposed on both sides of the MEA 10. The bipolar plates 20 include a conductive metal, carbon or the like, and function as current collectors, while providing oxygen and fuel to the catalytic layers of the MEAs 10.

Although only two unit cells 11 are shown in FIG. 8, the number of unit cells is not limited to two and a fuel cell may have several tens or hundreds of unit cells, depending on the required properties of the fuel cell.

Referring to FIG. 9, each MEA 10 includes an electrolyte membrane 100 according to the present embodiment, catalytic layers 110 and 110′ respectively disposed on either side of the electrolyte membrane 100 in the thickness direction thereof, first gas diffusion layers 121 and 121′ respectively stacked on the catalytic layers 110 and 110′, and second gas diffusion layers 120 and 120′ respectively stacked on the first gas diffusion layers 121 and 121′.

The catalytic layers 110 and 110′ function as a fuel electrode and an oxygen electrode each including a catalyst and a binder, and may further include a material that can increase the electrochemical surface area of the catalyst.

The first gas diffusion layers 121 and 121′ and the second gas diffusion layers 120 and 120′ may each be formed of a material such as, for example, carbon sheet or carbon paper. The first gas diffusion layers 121 and 121′ and the second gas diffusion layers 120 and 120′ diffuse oxygen and fuel supplied through the bipolar plates 20 to the entire surface of the catalytic layers 110 and 110′.

The fuel cell 1 including the MEAs 10 operates at a temperature of 100 to 300° C. Fuel such as hydrogen is supplied through one of the bipolar plates 20 into a first catalytic layer, and an oxidant such as oxygen is supplied through the other bipolar plate 20 into a second catalytic layer. Then, hydrogen is oxidized into protons in the first catalytic layer, and the protons are conducted to the second catalytic layer through the electrolyte membrane. Then, the protons electrochemically react with oxygen in the second catalytic layer to produce water and generate electrical energy. Moreover, hydrogen supplied as a fuel may be hydrogen produced by reforming hydrocarbons or alcohols. Oxygen supplied as an oxidant may be supplied in the form of air.

Hereinafter, a fuel cell 1 including the nanocomposite electrolyte membrane according to an embodiment of the present invention will be described below in detail. The nanocomposite electrolyte membrane may be used in any fuel cell 1 that includes an electrolyte membrane 100 containing a polymer electrolyte, such as a polymer electrolyte membrane fuel cell (PEMFC) using hydrogen as a fuel. The nanocomposite electrolyte membrane may be also used in a specific type of PEMFC. For example, the PEMFC can be a direct methanol fuel cell using a mixture vapor of methanol and water or an aqueous methanol solution as a fuel. The nanocomposite electrolyte membrane may be more useful in a direct methanol fuel cell using an aqueous methanol solution as a fuel, but the invention is not limited thereto.

According to an embodiment of the present invention, in a fuel cell 1 that includes a cathode in which oxygen is reduced, an anode in which a fuel is oxidized, and an electrolyte membrane 100 interposed between the cathode and the anode, the nanocomposite electrolyte membrane according to an embodiment of the present invention is used as the electrolyte membrane 100. The cathode includes a catalyst layer 110 that catalyzes the reduction of oxygen. The catalyst layer 110 includes a catalyst particle and a polymer having a cation exchange group. The catalyst may be, for example, a platinum (Pt)-carbon supported (Pt/C) catalyst.

The anode includes a catalyst layer 110′ that catalyzes the oxidation of a fuel, such as hydrogen, natural gas, methanol, or ethanol. The catalyst layer 110′ includes a catalyst particle and a polymer having a cation exchange group. The catalyst may be, for example, a Pt-supported carbon catalyst or a platinum-ruthenium (Pt—Ru)-carbon supported catalyst. The Pt—Ru-carbon supported catalyst is useful when an organic fuel, excluding hydrogen, is directly supplied to the anode.

The catalyst used in the cathode and the anode includes catalyst metal particles and a catalyst support. The catalyst support may be a solid particle such as carbon powder that has conductivity and micropores capable of supporting catalyst metal particles. Examples of the carbon powder may include carbon black, ketjen black, acetylene black, active carbon powder, carbon fiber powder, and any mixtures thereof. The polymer having a cation exchange group may be the polymer described above. The catalyst layers of the cathode and the anode contact the nanocomposite electrolyte membrane.

Each of the cathode and the anode may further include, in addition to the catalyst layer, a gas diffusion layer, 121 and 121′ respectively. The gas diffusion layers 121 and 121′ include a porous material having electrical conductivity. The gas diffusion layers 121 and 121′ act as current collectors and passages through which reactants and reaction products move. The gas diffusion layers 121 and 121′ may be formed of a carbon paper, for example, a water-repellent carbon paper. For example, a water-repellent carbon paper that is coated with the water-repellent carbon black layer may be used. The water-repellent carbon paper includes a hydrophobic polymer, such as polytetrafluoroethylene (PTFE), wherein the hydrophobic polymer is sintered. The use of a water-repellent material in the gas diffusion layers 121 and 121′ is to secure the passages of polar liquid reactants and gas reactants simultaneously. In the water-repellent carbon paper having the water-repellent carbon black layer, the water-repellent carbon black layer includes carbon black and a hydrophobic polymer, such as PTFE, as a hydrophobic binder, and is attached to one surface of the water-repellent carbon paper. The hydrophobic polymer of the water-repellent carbon black layer is sintered.

The cathode and the anode may be manufactured using various well-known methods, and thus will not be described in detail. According to the present embodiment, the fuel cell 1 may be a direct methanol fuel cell.

The glass transition temperature of the sulfonated polyarylene sulfone, the cross-linked product thereof, and the nanocomposite using the same is lowered and thus brittleness and solvent resistance thereof are improved. Accordingly when the nanocomposite electrolyte membrane formed using these materials is used, mechanical properties and ionic conductivity are improved, and crossover of a fuel to the cathode is reduced. Thus, a fuel cell having improved output and lifetime may be manufactured.

Hereinafter, one or more embodiments of the present invention will be described in detail with reference to the following examples. These examples are not intended to limit the purpose and scope of the one or more embodiments of the present invention.

### Synthesis Example 1

### Preparation of Alkylated Bisphenol A

Alkenes were mixed with diallyl bisphenol A by using the composition ratios illustrated in Table 1 below. Then, AIBN as a radical polymerization initiator and N-methylpyrrolidone as a solvent were added to the mixture and the mixture was reacted, thereby manufacturing alkylated bisphenol A of Formula 1.

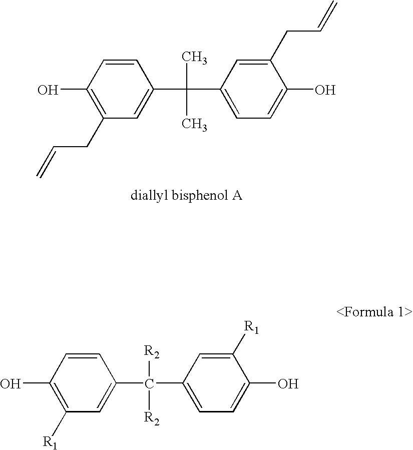

where R₁ is octyl, dodecyl, or eicosyl and R₂ is methyl.

TABLE 1

| | | | Amount |
|---|---|---|---|
| | | Amount | of diallyl |
| | | of alkene | bisphenol |
| No. | alkenes | (mole) | A (mole) |
| bisphenol A | 1-octene | 2 | 1 |
| dipentadecyll | 1-dodecene | 2 | 1 |
| ditricosyl bisphenol | 1-eicosene | 2 | 1 |

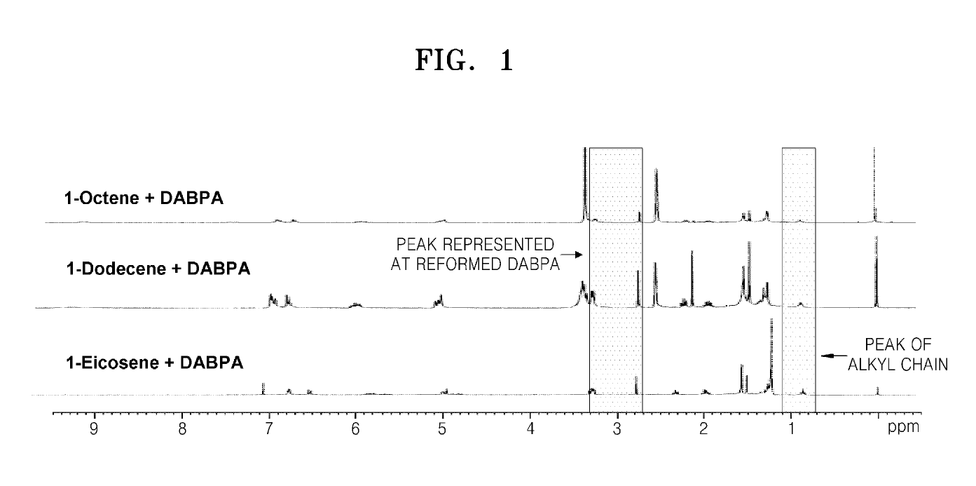

A nuclear magnetic resonance (NMR) analysis was performed on diundecyl bisphenol A, dipentadecyl bisphenol A, and tricosyl bisphenol A and structures thereof were found with reference to FIG. 1.

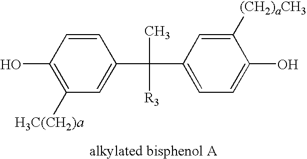

### Comparative Example 1 and Examples 1 to 9

In accordance with the conditions shown in Table 2 below, alkylated bisphenol As were prepared.

TABLE 2

| | | Amount of | | reaction | | |
|---|---|---|---|---|---|---|
| | Initiator | diallyl bisphenol | | time | | |
| Sample | (mol) | A (mol) | Alkene (mol) | (Hr) | solvent | Yield (%) |
| Comparative | AIBN(0.6) | 1 | dodecene (2) | 24 | toluene | 0 |
| Example 1 | | | | | | |
| Example 1 | AIBN(0.6) | 1 | dodecene (2) | 48 | toluene | 0 |
| Example 2 | AIBN(0.6) | 1 | dodecene (2) | 72 | toluene | 0 |
| Example 3 | BPO(0.6) | 1 | dodecene (2) | 24 | toluene | 0 |
| Example 4 | AIBN(0.6) | 1 | dodecene (2) | 24 | toluene | 0 |
| Example 5 | AIBN(0.6) | 1 | dodecene (2) | 24 | DMF | 50 |
| Example 6 | AIBN(0.6) | 1 | dodecene (2) | 24 | NMP | 55 |
| Example 7 | AIBN(2.0) | 1 | octene (2) | 24 | NMP | 82 |
| Example 8 | AIBN(2.0) | 1 | dodecene (2) | 24 | NMP | 82 |
| Example 9 | AIBN(2.0) | 1 | eicosene (2) | 24 | NMP | 83 |

As illustrated in Table 2 above, reactivities are different from each other according to the type of reaction solvents. That is, the polar solvent, NMP shows greater alkylation efficiency than non-polar toluene.

### Synthesis Example 2

### Preparation of Sulfonated Polyarylene Sulfone

4,4′-dichlorodiphenyl sulfone (DCDPS), sodium sulfonated 4,4′-dichlorodiphenyl sulfone (Na-sDCDPS), and alkylated bisphenol-A obtained according to Synthesis Example 1 were dissolved in 70 ml of N-methylpyrrolidone according to the composition ratios in Table 3 below, and sodium hydrogen carbonate (1 mol), toluene, and N-methylpyrrolidone were added thereto. Then, water generated by refluxing the mixture at about 150° C. was removed from the mixture and the mixture was left to stand for about 6 hours or more at about 180° C. Then, the mixture was condensation polymerized to obtain sulfonated polyarylene sulfone (refer to Comparative Example 2 and Examples 10 to 20).

TABLE 3

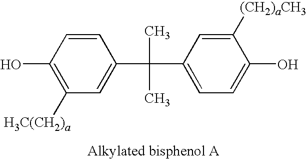

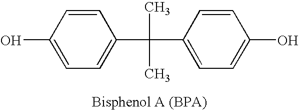

| | | Mixing molar | *SD value % |
|---|---|---|---|
| | | ratio of | (Mixing molar |
| | | bisphenol A and | ratio |
| | alkylated bisphenol | alkylated | of s-DCDPS |
| Sample | A | bisphenol A | and DCCPS) |
| Comparative | — | 100:0 | 30% (30:70) |
| Example 2 | | | |
| Example 10 | undecyl bisphenol A | 0:100 | 30% (30:70) |
| | (a = 7) | | |
| Example 11 | dipentadecyl | 90:10 | 30% (30:70) |
| | bisphenol A (a = 11) | | |
| Example 12 | dipentadecyl | 75:25 | 30% (30:70) |
| | bisphenol A (a = 11) | | |
| Example 13 | dipentadecyl | 50:50 | 30% (30:70) |
| | bisphenol A (a = 11) | | |
| Example 14 | dipentadecyl | 25:75 | 30% (30:70) |
| | bisphenol A (a = 11) | | |
| Example 15 | dipentadecyl | 0:100 | 30% (30:70) |
| | bisphenol A (a = 11) | | |
| Example 16 | ditricosyl bisphenol A | 90:10 | 30% (30:70) |
| | (a = 15) | | |
| Example 17 | ditricosyl bisphenol A | 75:25 | 30% (30:70) |
| | (a = 15) | | |
| Example 18 | ditricosyl bisphenol A | 50:50 | 30% (30:70) |
| | (a = 15) | | |
| Example 19 | ditricosyl bisphenol A | 25:75 | 30% (30:70) |
| | (a = 15) | | |
| Example 20 | ditricosyl bisphenol A | 0:100 | 30% (30:70) |
| | (a = 15) | | |

*The SD value denotes the degree of sulfonation and represents the number of moles of s-DCDPS as a percentage of the total number of moles of s-DCDPS and DCCPS. For example, when the SD value is 30%, the s-DCDPS and the DCCPS are used in the mixing molar ratio of 30:70.

When the mixing molar ratio of bisphenol A and alkylated bisphenol A was 100:0 in Table 3, sulfonated polyarylene sulfone of Formula 5a below was obtained.

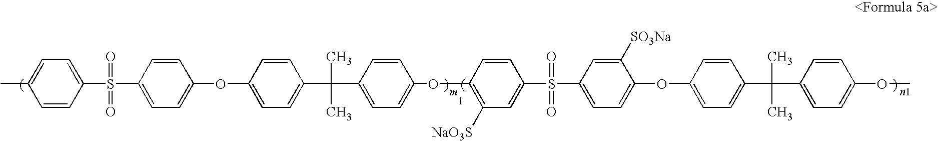

where m1 is about 0.7 and n1 is about 0.3.

When the mixing molar ratios of bisphenol-A and alkylated bisphenol A were respectively 90:10, 75:25, 50:50, and 25:75, sulfonated polyarylene sulfone including four repeating units represented by Formulas 5b and 5c below were obtained.

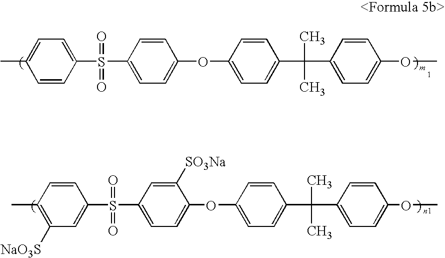

where m1 is from about 0.05 to about 0.99 and n1 is from about 0.01 to about 0.95.

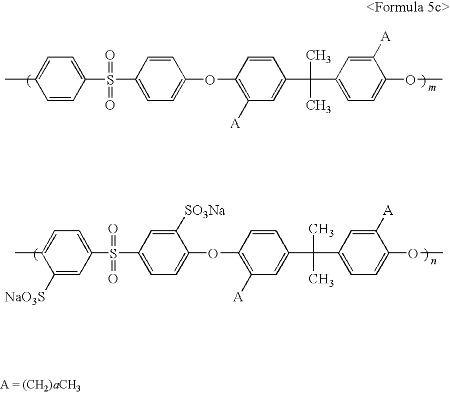

where m is from about 0.05 to about 0.99 and n1 is from about 0.01 to about 0.95.

When the mixing molar ratio of bisphenol A and alkylated bisphenol A was 0:100 in Table 3, sulfonated polyarylene sulfone of Formula 5 was obtained.

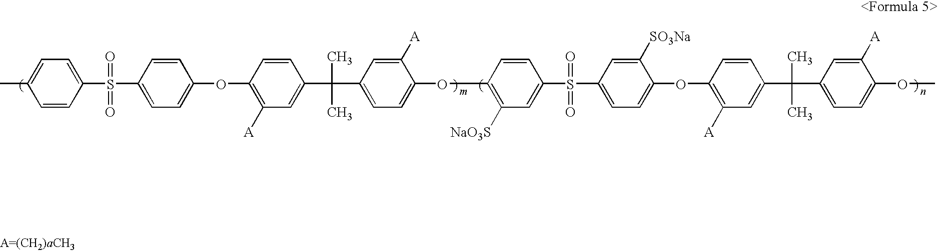

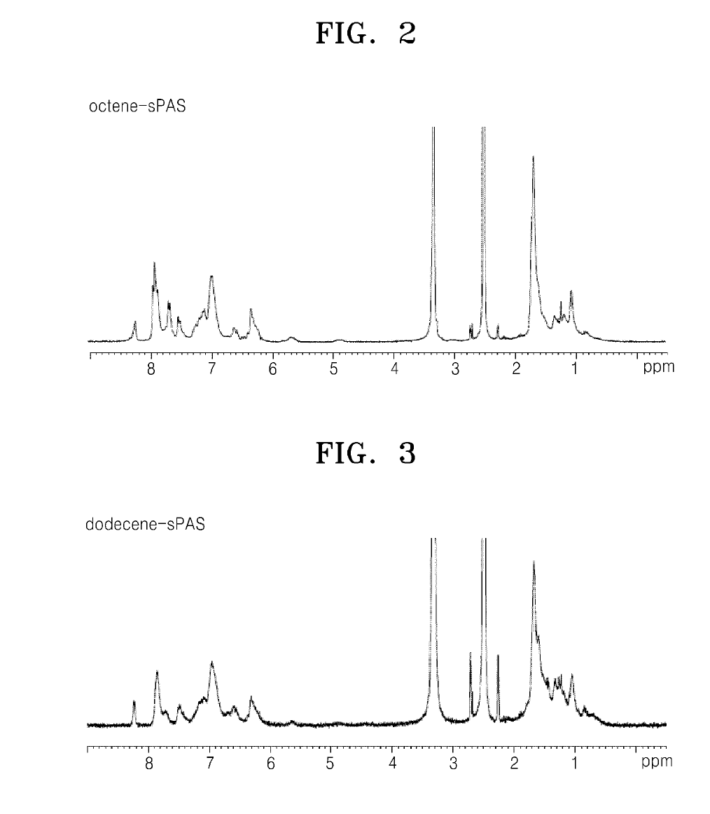

NMR spectrum analysis was performed on the sulfonated polyarylene sulfones obtained according to Examples 10 and 11 and the results are shown in FIGS. 2 and 3. Accordingly, the structure of the sulfonated polyarylene sulfone was found through the NMR results.

The number average molecular weight (Mn), weight average molecular weight (Mw), and molecular weight distribution (Mw/Mn) were measured for the sulfonated polyarylene sulfones prepared according to Comparative Example 2 and Examples 10 through 20 by using Gel Permeation Chromatograph (GPC) and the results are shown in Table 4 below.

TABLE 4

| | Number | | | |
|---|---|---|---|---|
| | average | | Molecular | |
| | molecular | Weight average | weight | |
| | weight | molecular weight | distribution | |
| Sample | (Mₙ) | (M_{w}) | (M_{w}/Mₙ) | T_{g} |
| Comparative | 575,069 | 1,011,000 | 1.759 | 203.81° C. |
| Example 2 | | | | |
| Example 10 | 693,427 | 1,170,000 | 1.688 | 173.7° C. |
| Example 11 | 718,888 | 1,250,000 | 1.738 | 185.48° C. |
| Example 12 | 579,495 | 1,018,000 | 1.756 | 191.1° C. |
| Example 13 | 647,382 | 1,152,000 | 1.779 | 175.2° C. |
| Example 14 | 679,972 | 1,229,000 | 1.807 | 154.6° C. |
| Example 15 | 712,561 | 1,306,000 | 1.833 | 156.7° C. |
| Example 16 | 1,086,000 | 1,933,000 | 1.780 | 176.86° C. |
| Example 17 | 501,170 | 984,299 | 1.964 | 157.1° C. |
| Example 18 | 563,550 | 1,047,650 | 1.870 | 145.7° C. |
| Example 19 | 625,930 | 1,111,000 | 1.775 | 138.5° C. |
| Example 20 | 1,337,000 | 2,571,000 | 1.923 | 139.9° C. |

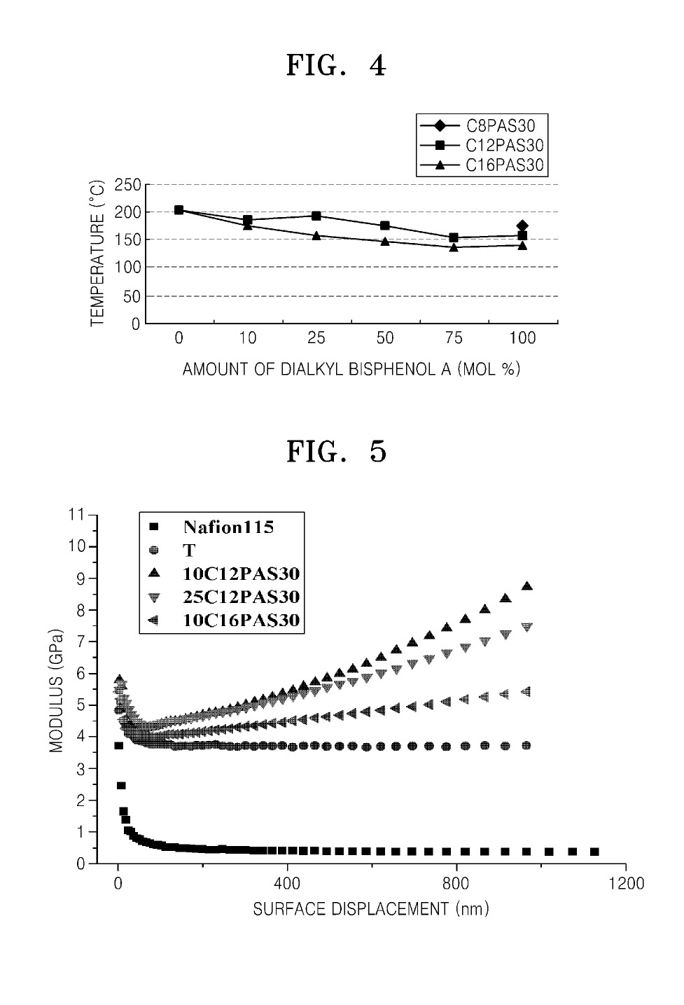

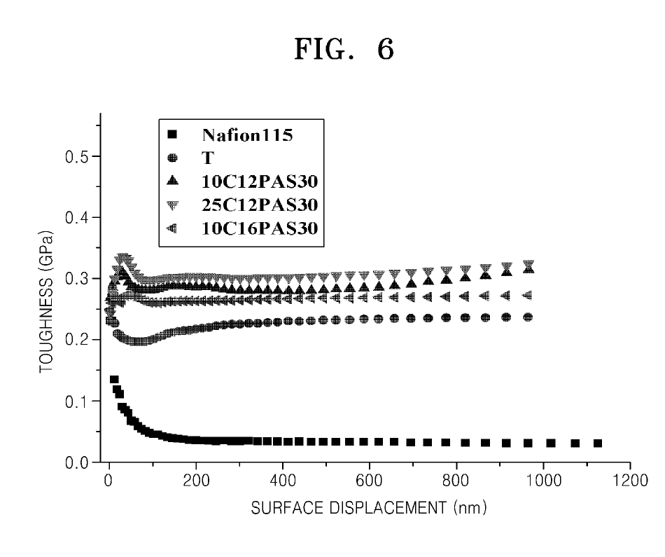

The sulfonated polyarylene sulfones prepared according to Examples 10 through 20 were analyzed using differential scanning calorimetry (DSC), and the results are shown in Table 4 and FIG. 6. Referring to FIG. 4 and Table 4, the glass transition temperature decreases as a function of the chain length of the alkenyl group that is reacted to form the alkylated bisphenol A.

The compression modulus and hardness of each of the sulfonated polyarylene sulfones prepared according to Examples 11, 12 and 16 were measured, and the results are shown in FIGS. 5 and 6. Referring to FIGS. 5 and 6, the sulfonated polyarylene sulfone prepared using alkylated bisphenol A has a high compression modulus and excellent toughness (value in which toughness curve is integrated by surface displacement) and this indicates that the problem of membrane thinning of an elastomeric polymer such as NAFION® (DuPont Company) is reduced.

The contact angles of the sulfonated polyarylene sulfones prepared according to Comparative Example 2 and Examples 11 through 15 were measured. The contact angles were analyzed using a contact angle analyzer. It can be seen from analyzing the contact angle that as the amount of dialkyl bisphenol A increases, hydrophobicity increases. Thus, the water contact angle with respect to the surface of the sulfonated polyarylene sulfone also increases.

The sulfonated polyarylene sulfones prepared according to Examples 16 and 19 were analyzed using atomic force microscopy (AFM. Specifically, contact angles were evaluated using a tapping mode method by using the AFM. In this case, bright portions of resulting AFMs indicate high transition temperatures (Tg) regions and dark portions indicate low Tg regions. Here, as the amount of dialkyl bisphenol A increases, the soft (high Tg) region increases.

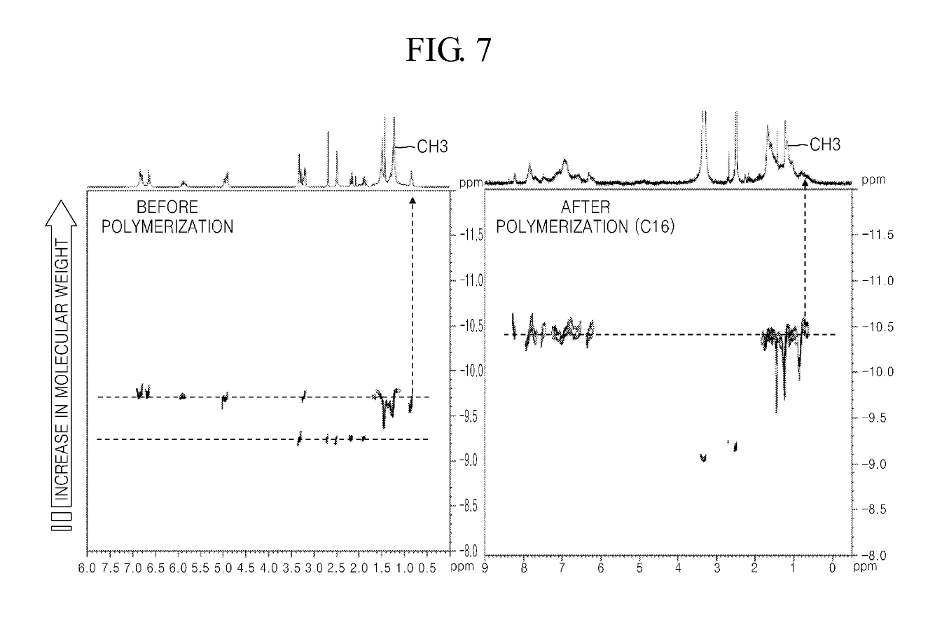

The sulfonated polyarylene sulfone prepared according to Example 16 was analyzed before and after polymerization using diffusion ordered spectroscopy (DOSY), and the results are shown in FIG. 7. The sulfonated polyarylene sulfone prepared according to the conditions of Table 2 was used to prepare an electrolyte membrane and the conductivity of the electrolyte membrane was measured at a temperature of about 25° C. As a result, it was found that the electrolyte membranes of Examples 11 and 16 had higher ionic unit conductivity than that of Comparative Example 2.

In addition, the sulfonated polyarylene sulfone and the cross-linked product thereof obtained as above were used to form an electrolyte membrane and the electrolyte membrane was immersed in NMP for about 72 hours. Then the state of the electrolyte membrane was analyzed.

The electrolyte membrane formed from the sulfonated polyarylene sulfone was dissolved in NMP and thus a membrane form was not maintained. However, in the electrolyte membrane formed from the cross-linked product of the sulfonated polyarylene, swelling by NMP is shown but the membrane form is maintained. Accordingly, the cross-linked product of the sulfonated polyarylene sulfone has solvent resistance.

TABLE 5

| | | | | Initial | |
|---|---|---|---|---|---|
| | | | | con- | |
| | | thickness | | centration | |
| | transmissivity | (Wet) | area | of MeOH | Vb |
| sample | (cm²/s) | (μm) | (1 cm²) | (3M) | (35 cm²) |
| Example | 8.86 × 10⁻⁷ | 45 | 1 | 3 | 35 |
| 11 | | | | | |
| Example | 7.36 × 10⁻⁷ | 35 | 1 | 3 | 35 |
| 16 | | | | | |
| Nafion | 1.87 × 10⁻⁶ | 140 | 1 | 3 | 35 |
| 115 | | | | | |

TABLE 6

| | DCDPS/s- | | | Ionic unit |
|---|---|---|---|---|
| | DCDPS | Thickness (Wet) | | conductivity |
| sample | ratio | (μm) | Area (1 cm²) | [S/cm²] |
| Example 11 | 3:7 | 45 | 1 | 13.3 |
| Example 16 | 3:7 | 35 | 1 | 8.4 |
| Nafion115 | 3:7 | 140 | 1 | 6.7 |

As shown in Tables 5 and 6, MeOH transmissivity is lower in Examples 11 and 16 than that of Nafion 115 and ionic unit conductivity of Examples 11 and 16 is also higher than that of Nafion 115.

As described above, according to the one or more of the above embodiments of the present invention, the alkylated bisphenol-based compound is used to form the sulfonated polyarylene sulfone and the clay-sulfonated polyarylene sulfone nanocomposite and thus hydrophobicity is increased and the glass transition temperature of the sulfonated polyarylene sulfone is adjusted.

Although a few embodiments of the present invention have been shown and described, it would be appreciated by those skilled in the art that changes may be made in these embodiments without departing from the principles and spirit of the invention, the scope of which is defined in the claims and their equivalents.

What is claimed is:

1. An alkylated bisphenol-based compound represented by Formula 1: 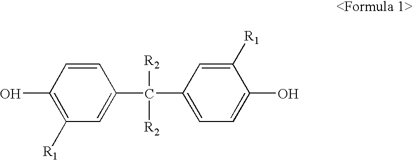
wherein R₁ is a C1 to C30 alkyl group, and
R₂ is a hydrogen atom or a C1 to C5 alkyl group.

2. The alkylated bisphenol-based compound of claim 1, wherein R₁ is undecyl (—(CH₂)₁₀CH₃), pentadecyl (—(CH₂)₁₄CH₃), heneicosyl (—(CH₂)₂₀CH₃), or tricosyl (—(CH₂)₂₂CH₃).

3. The alkylated bisphenol-based compound of claim 1, wherein:
the alkylated bisphenol-based compound of Formula 1 is a compound represented by Formula 2, and 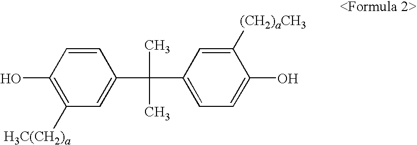
a is an integer from about 4 to about 22.

4. A sulfonated polyarylene sulfone comprising a first repeating unit represented by Formula 3 and a second repeating unit represented by Formula 4: 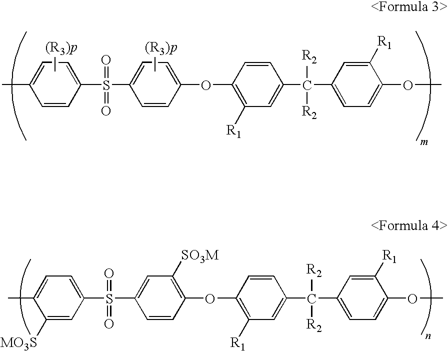
wherein R₁ is a C1 to C30 alkyl group,
R₂ is a hydrogen atom or a C1 to C5 alkyl group,
each R₃ is the same as r or different from other R₃'s and is independently a C1-C10 alkyl group, a C2-C10 alkenyl group, a phenyl group or a nitro group,
p is an integer from about 0 to about 4,
M is sodium (Na), potassium (K), or hydrogen (H), and
m is from about 0.01 to about 0.99, and n is from about 0.01 to about 0.99.

5. The sulfonated polyarylene sulfone of claim 4, wherein the degree of the polymerization of the sulfonated polyarylene sulfone is in the range of about 5 to about 3,500.

6. The sulfonated polyarylene sulfone of claim 4, wherein R₁ is undecyl (—(CH₂)₁₃CH₃), pentadecyl (—(CH₂)₁₄CH₃), heneicosyl (—(CH₂)₂₀CH₃), or tricosyl (—(CH₂)₂₂CH₃).

7. The sulfonated polyarylene sulfone of claim 4, wherein:
the sulfonated polyarylene sulfone is a repeating unit represented by Formula 5, 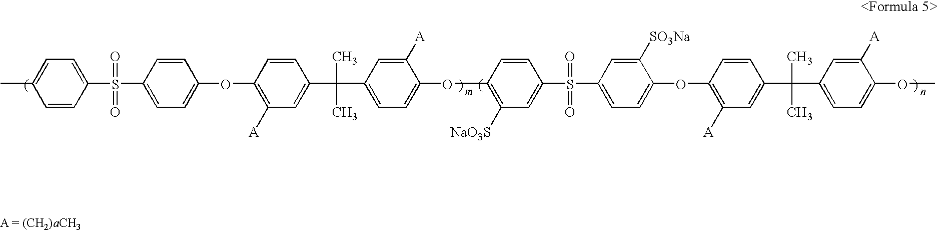
m is from about 0.01 to about 0.99,
n is from about 0.01 to about 0.99,
the degree of polymerization is about 5 to about 3500, and
a is an integer from about 4 to about 22.

8. The sulfonated polyarylene sulfone of claim 4, wherein:
the sulfonated polyarylene sulfone is a polymer comprising repeating units represented by Formulas 5b and 5c below, 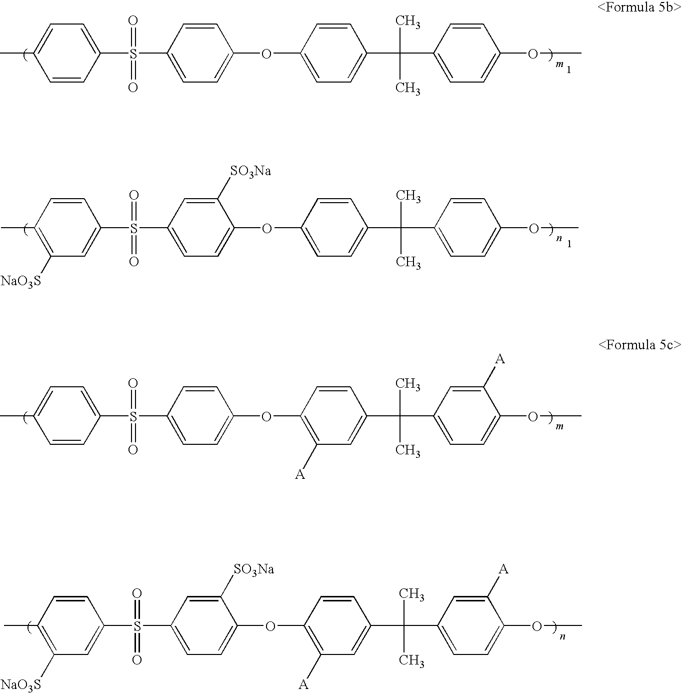
m1 is from about 0.01 to about 0.99,
n1 is from about 0.01 to about 0.99,
m is from about 0.01 to about 0.99,
n is from about 0.01 to about 0.99,
a is an integer from about 4 to about 22, and
the degree of polymerization is about 5 to about 3500.

9. The sulfonated polyarylene sulfone of claim 4, wherein the sulfonated polyarylene sulfone has a degree of sulfonation of about 10 to about 50%.

10. The sulfonated polyarylene sulfone of claim 4, further comprising at least one repeating unit selected from the group consisting of a third repeating unit represented by Formula 3a and a fourth repeating unit represented by Formula 4a: 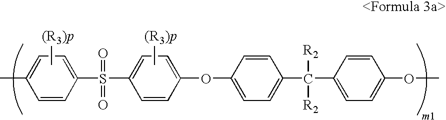
m1 is from about 0.01 to about 0.99. 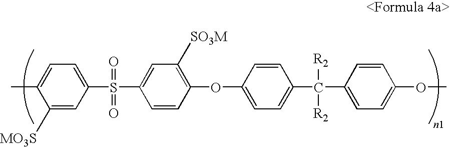
wherein R₂ is a hydrogen atom or a C1 to C5 alkyl group,
each R₃ is the same as or different from other R₃'s and is a C1-C10 alkyl group, a C2-C10 alkenyl group, a phenyl group or a nitro group,
p is an integer from about 0 to about 4,
M is sodium (Na), potassium (K), or hydrogen (H), and
M1 and n1 are each from about 0.01 to about 0.99.

11. A method of preparing an alkylated bisphenol-based compound represented by Formula 1, the method comprising of adding a C2-C30 alkene to a compound represented by Formula 6 below to obtain the alkylated bisphenol-based compound: 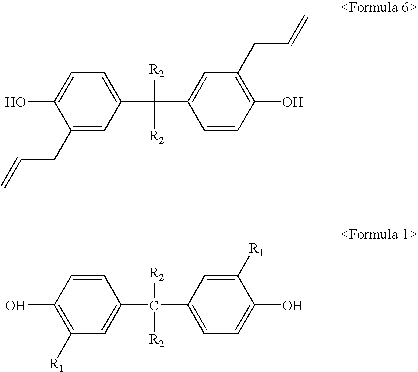
wherein R₁ is a C1 to C30 alkyl group; and
R₂ is a hydrogen atom or a C1 to C5 alkyl group.

12. The method of claim 11, wherein the C2-C30 alkene comprises at least one selected from the group consisting of 1-octene, 1-dodecene, 1-octadecene and 1-eicosene.

13. The method of claim 11, wherein the addition is performed in the presence of a radical polymerization initiator.

14. The method of claim 13, wherein the radical polymerization initiator comprises azobisisobutyronitrile (AIBN) and benzyl peroxide.

15. A method of preparing sulfonated polyarylene sulfone comprising a first repeating unit represented by Formula 3 below and a second repeating unit represented by Formula 4 below, the method comprising performing polymerization of an alkylated bisphenol-based compound represented by Formula 1 below, a compound represented by Formula 7 below, and a compound represented by Formula 8 below, wherein: 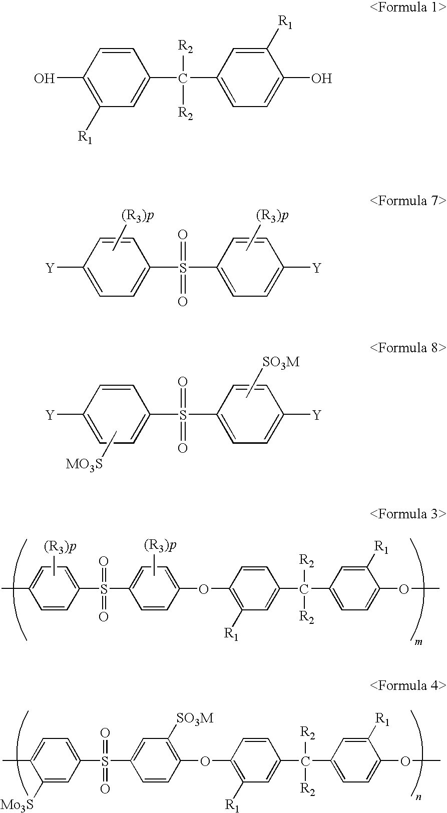
wherein R₁ is a C1 to C30 alkyl group,
R₂ is a hydrogen atom or a C1 to C5 alkyl group,
each R₃ is the same as or different from other R₃'s and is a C1-C10 alkyl group, a C2-C10 alkenyl group, a phenyl group or a nitro group,
p is an integer from about 0 to about 4,
Y is chlorine (Cl), fluorine (F), bromine (Br), or iodine (I),
M is sodium (Na), potassium (K), or hydrogen (H),
m is from about 0.01 to about 0.99, and
n is from about 0.01 to about 0.99.

16. The method of claim 15, further comprising adding a bisphenol-based compound represented by Formula 9, 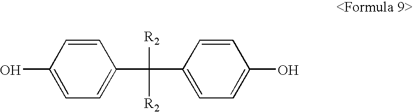
wherein R₂ is a hydrogen atom or a C1 to C5 alkyl group.

17. The method of claim 16, wherein the polymerization is performed at a temperature of about 100 to about 190° C.

18. A fuel cell comprising:
a cathode;
an anode and
an electrolyte membrane comprising sulfonated polyarylene sulfone and disposed between the cathode and the anode, wherein:
the sulfonated polyarylene sulfone comprises a first repeating unit represented by Formula 3 below and a second repeating unit represented by Formula 4 below, 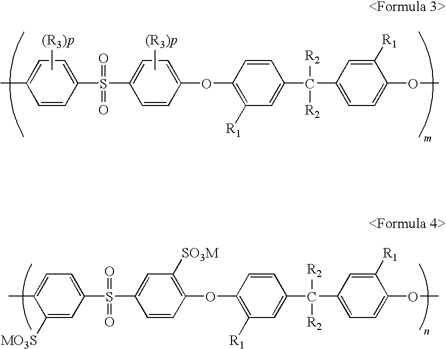
R₁ is a C1 to C30 alkyl group,
R₂ is a hydrogen atom or a C1 to C5 alkyl group,
each R₃ is the same as or different from other R₃'s and is a C1-C10 alkyl group, a C2-C10 alkenyl group, a phenyl group or a nitro group,
p is an integer from about 0 to about 4,
M is sodium (Na), potassium (K), or hydrogen (H),
m is from about 0.01 to about 0.99, and
n is from about 0.01 to about 0.99.

19. The fuel cell of claim 18, wherein the electrolyte membrane further comprises a clay and sulfonated polyarylene sulfone nanocomposite.

20. The fuel cell of claim 18, wherein the sulfonated polyarylene sulfone further comprises at least one repeating unit selected from the group consisting of a third repeating unit represented by Formula 3a below and a fourth repeating unit represented by Formula 4a below. 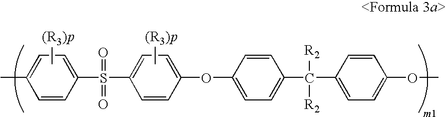
wherein R₂ is a hydrogen atom or a C1 to C5 alkyl group,
m1 is from about 0.01 to about 0.99 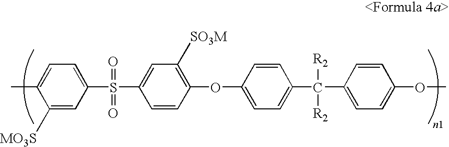
wherein R₂ is a hydrogen atom or a C1 to C5 alkyl group,
each R₃ is the same as or different from other R₃'s and is a C1-C10 alkyl group, a C2-C10 alkenyl group, a phenyl group or a nitro group,
p is an integer from about 0 to about 4,
M is sodium (Na), potassium (K), or hydrogen (H), and
n1 is from about 0.01 to about 0.99.

21. The sulfonated polyarylene sulfone of claim 4, wherein the sulfonated polyarylene sulfone is a cross-linked sulfonated polyarylene sulfone.

22. The sulfonated polyarylene sulfone of claim 4, wherein the sulfonated polyarylene sulfone is mixed with a clay to form a clay-sulfonated polyarylene sulfone nanocomposite.

23. An electrolyte membrane for a fuel cell comprising sulfonated polyarylene sulfone containing a first repeating unit represented by Formula 3 below and a second repeating unit represented by Formula 4 below, 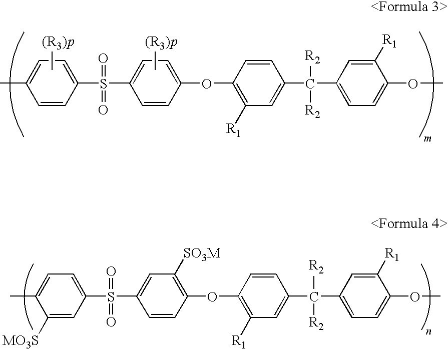
wherein:
R₁ is a C1 to C30 alkyl group,
R₂ is a hydrogen atom or a C1 to C5 alkyl group,
each R₃ is the same as or different from other R₃'s and is a C1-C10 alkyl group, a C2-C10 alkenyl group, a phenyl group or a nitro group,
p is an integer from about 0 to about 4,
M is sodium (Na), potassium (K), or hydrogen (H),
m is from about 0.01 to about 0.99, and
n is from about 0.01 to about 0.99.

24. The electrolyte membrane of claim 23, wherein the sulfonated polyarylene sulfone is a cross-linked sulfonated polyarylene sulfone.

25. The electrolyte membrane of claim 23, wherein the sulfonated polyarylene sulfone is mixed with a clay to form a clay-sulfonated polyarylene sulfone nanocomposite.

26. The electrolyte membrane of claim 23, wherein the sulfonated polyarylene sulfone further comprises at least one repeating unit selected from the group consisting of a third repeating unit represented by Formula 3a below and a fourth repeating unit represented by Formula 4a below. 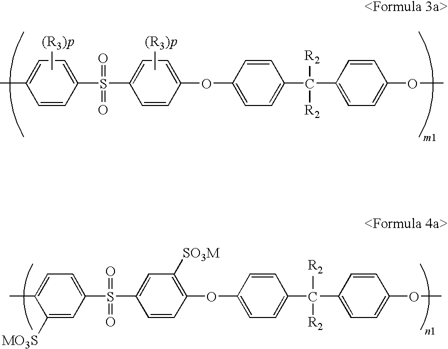
wherein:
R₂ is a hydrogen atom or a C1 to C5 alkyl group,
each R₃ is the same as or different from other R₃'s and is a C1-C10 alkyl group, a C2-C10 alkenyl group, a phenyl group or a nitro group,
p is an integer from about 0 to about 4,
M is sodium (Na), potassium (K), or hydrogen (H),
m1 is from about 0.01 to 0.99, and
n1 is from about 0.01 to about 0.99.
